(19) United States
(12) Patent Application Publication
(10) Pub. No.: US 2022/0387126 A1
(43) Pub. Date: Dec. 8, 2022

(54) HEAD STABILIZATION SYSTEM WITH SENSING FEATURES

(71) Applicant: pro med instruments GmbH, Freiburg Im Breisgau (DE)

(72) Inventors: Severin Moosmann, Friesenheim (DE); Jan H. Mertens, Freiburg (DE); Roman Maier, Scheer (DE); Matthias E. Schuele, Freiburg (DE)

(21) Appl. No.: 17/834,172

(22) Filed: Jun. 7, 2022

Related U.S. Application Data
(60) Provisional application No. 63/208,240, filed on Jun. 8, 2021.
(60) Provisional application No. 63/208,255, filed on Jun. 8, 2021.

(51) Int. Cl. A61B 90/14; A61B 5/11; A61B 5/00; A61B 90/00

(52) U.S. Cl. CPC A61B 90/14; A61B 5/1126; A61B 5/746; A61B 90/36; A61B 2090/365

(57) ABSTRACT

A head stabilization system useable for stabilizing a head of a patient during a medical procedure includes a sensor assembly and a connection assembly connected with a head fixation device. The sensor assembly includes one or more sensors positioned on the head fixation device for detecting one or more characteristics of the head fixation device. The connection assembly receives and processes the detected characteristic to provide feedback of the detected characteristic. The head fixation device can be adjusted or modified based on the provided feedback.

### PRIORITY

This application claims priority to U.S. Provisional Patent Application Ser. No. 63/208,240, filed Jun. 8, 2021, entitled “Head Stabilization System with Sensing Features,” and U.S. Provisional Patent Application Ser. No. 63/208,255, filed Jun. 8, 2021, entitled “Head Stabilization System with Sensing Features,” the disclosures of which are incorporated by reference herein.

### BACKGROUND

During certain medical procedures it may be necessary or desirable to stabilize all or a portion of a patient such that the patient or portion of the patient is immobilized. In certain neurological procedures the portion stabilized may include the head and/or neck of the patient. Certain devices and methods may be used to stabilize a certain portion of the patient. For example, a skull clamp is a type of head stabilization device that may be used to stabilize the head and/or neck of the patient. Furthermore, it may also be necessary or desirable to use various imaging modalities to obtain images of the patient before, during, and/or after a procedure.

Skull clamps are typically manually adjusted relative to a skull of the patient to apply a sufficient amount of force onto the skull to stabilize the patient. If the skull clamp is not sufficiently positioned relative to the skull, slippage can occur during the medical procedure that can cause problems. Moreover, overtightening of the skull clamp against the skull can result in an unintentional fracture of the bone or other injury to the patient. Accordingly, it is desirable to have a skull clamp system for supporting and stabilizing the head and/or neck of a patient during certain medical procedures that is able to detect and/or provide feedback about the integrity of the stabilization to avoid possible problems during the medical procedure.

While a variety of head stabilization devices and method of use of the same have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a schematic view of an exemplary head stabilization system.

FIG. 2 depicts a partial front view of a first pin holder assembly of a skull clamp of the head stabilization system of FIG. 1.

FIG. 3 depicts a partial cross section view of the first pin holder assembly of FIG. 2.

FIG. 4 depicts a partial front view of a second pin holder assembly of the skull clamp of the head stabilization system of FIG. 1.

FIG. 5 depicts a partial cross section view of the second pin holder assembly of FIG. 4.

FIG. 6 depicts a flowchart of an exemplary method of operating an exemplary head stabilization system such as that shown in FIG. 1.

FIG. 7 depicts a schematic view of another exemplary head stabilization system.

FIG. 8 depicts a flowchart of an exemplary method of operating an exemplary head stabilization system such as that shown in FIG. 7.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Exemplary Head Stabilization System

An exemplary head stabilization system comprises a sensor assembly and a connection assembly connected with a head fixation device. The sensor assembly comprises one or more sensors positioned on one or more components of the head fixation device and is configured to detect one or more characteristics of the head fixation device. The connection assembly comprises a data processing unit that is configured to receive and process data detected by the sensor assembly. For instance, the data processing unit can determine whether data received from the sensor assembly has reached and/or exceeded a predetermined or threshold value, which can indicate an impending compromise to the stabilization using the head fixation device. The connection system can then be configured to communicate feedback to a user and/or manufacturer based on the detected characteristic. The head fixation device can be adjusted based on the feedback to avoid possible problems during a medical procedure.

A. Exemplary Head Fixation Device

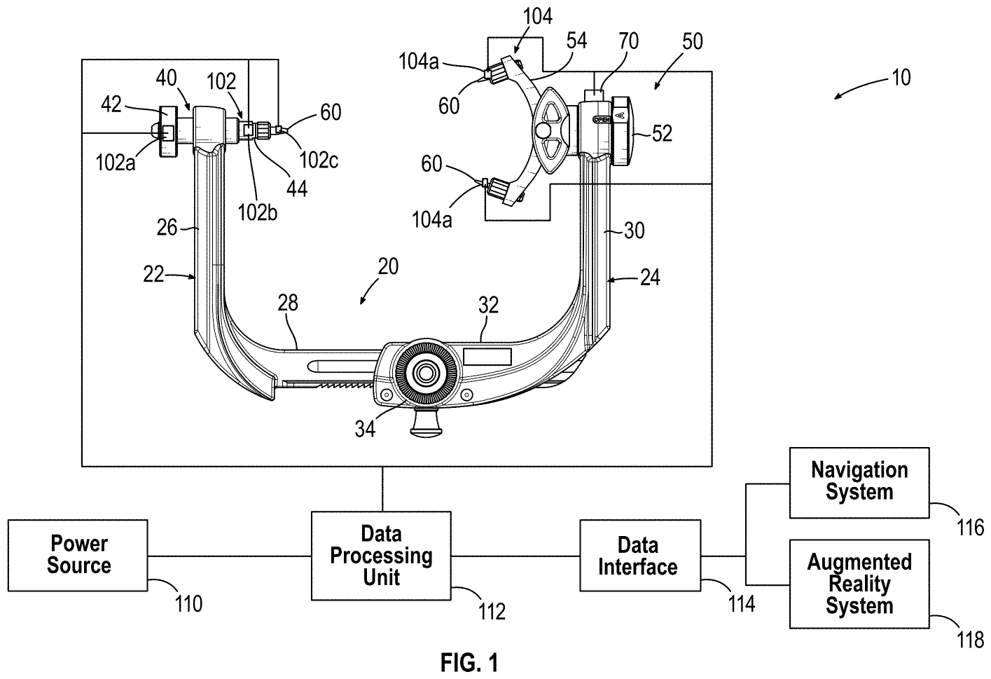

FIG. 1 illustrates an exemplary head stabilization system (10) having sensor features with an exemplary head stabilization or fixation device (20). Throughout the specification the term “HFD” is used interchangeably with the terms “head stabilization device,” “head fixation device,” or “skull clamp.” In the illustrated versions here HFD (20) has the shape or form of a skull clamp. While the present example illustrates the HFD as a U-shaped skull clamp, the teachings herein may be applied to other forms of HFDs as will be understood by those of ordinary skill in the art in view of the teachings herein. Skull clamp (20) can be made from a composite material, a polymer material (e.g., polyether ether ketone (PEEK), polytetrafluoroethylene (PTFE), etc.), and/or a metal material (e.g., aluminum, stainless steel, titanium, etc.). Skull clamp (20) comprises a first arm (22) and a second arm (24). First arm (22) is connectable with second arm (24) to form skull clamp (20) having a U-shape. First arm (22) comprises an upright portion (26) and a lateral portion (28). Similarly, second arm (24) comprises an upright portion (20) and a lateral portion (22). Skull clamp (20) is adjustable to accommodate a variety of head sizes by translating first arm (22) relative to second arm (24) or vice versa. Skull clamp (20) is further connectable to other structures, such as a positioning adapter or a base unit that is further connectable with an operating table, etc., by way of an attachment interface (34). As shown in the present example of FIG. 1, upright portion (26) of first arm (22) connects with a first pin holder assembly (40), while upright portion (30) of second arm (24) connects with a second pin holder assembly (50).

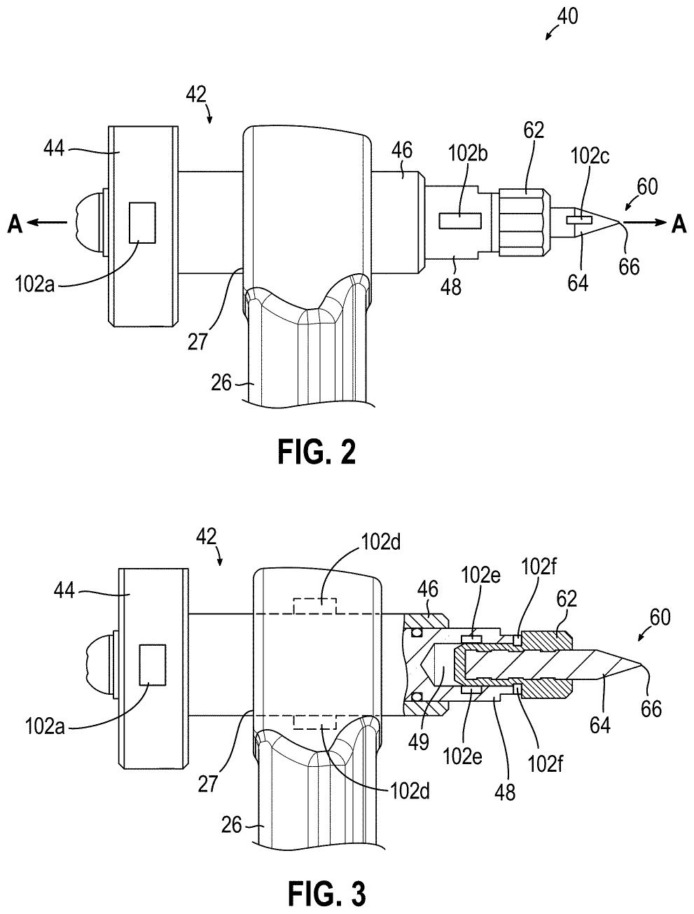

Referring to FIGS. 2-3, first pin holder assembly (40) comprises a torque screw (42) configured to adjust an amount of clamping force skull clamp (20) applies to the head of the patient. Torque screw (42) extends through a bore in upright portion (26) of arm (22) along a longitudinal axis (A). Torque screw (42) comprises an actuator in the form of a wheel (44), a sleeve (46), an internal spring (not shown), and an elongated member (48). Sleeve (46) engages with a bore (27) in upright portion (26) of arm (22). As wheel (44) is rotated, a pinning force applied by torque screw (42) increases or decreases depending on the direction of rotation of wheel (44). In view of the teaching herein, other ways to modify or use torque screw (42), or another similar structure, to control an amount of pinning force applied will be apparent to those of ordinary skill in the art.

At its distal end, elongated member (48) selectively retains a pin (60), which includes a housing (62) and a pin tip (64). In use, at least a distal tip (66) of pin (60) contacts the head of the patient. When wheel (44) is rotated in a first direction, the spring within torque screw (42) is compressed and thereby exerts an increased force on pin (60) in a direction toward the patient's head. Force applied by pin (60) to the head of the patient can be decreased by rotation of wheel (44) in the opposite direction. Accordingly, as wheel (44) is rotated, a pinning force applied by torque screw (42) and/or pin (60) increases or decreases depending on the direction of rotation of wheel (44).

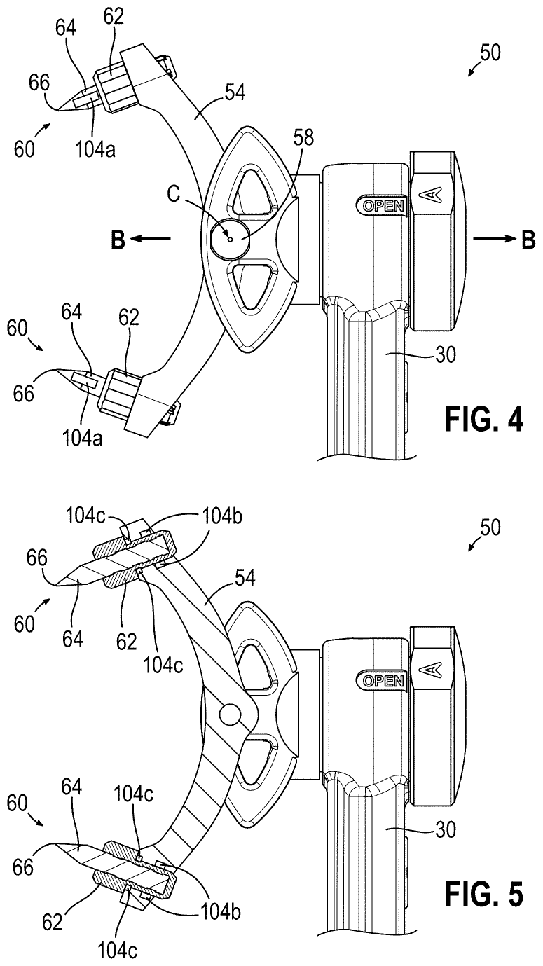

Referring to FIGS. 4-5, second pin holder assembly (50) connects with upright portion (30) of arm (24) as shown. Pin holder assembly (50) comprises rocker arm (54) that selectively retains a pair of pins (60), which each include housing (62) and pin tip (64). Pin holder assembly (50) is rotatably adjustable about an axis B extending through a bore in upright portion (30). This rotation can be selectively controlled such that in some versions the rotatable position of pin holder assembly (50) can be locked in position or unlocked for adjustment. In some other versions, pin holder assembly (50) is rotatably adjustable about axis B and is secured in its rotational position based on the force applied to the patient's head when clamping. In view of the teachings herein, various ways to configure pin holder assembly (50) to provide locking and unlocking states for rotational adjustment will be apparent to those of ordinary skill in the art. Pin holder assembly (50) is also configured such that rocker arm (54) is pivotably adjustable about an axis C defined longitudinally by pin (58). Regardless of the rotational or pivotal adjustments, the force applied to the head of the patient via torque screw (42) of pin holder assembly (40) as described above, causes force to also be applied to the head of the patient from pin holder assembly (50) and its pins (60) that are configured to contact the head of the patient. With the configuration described above for skull clamp (20), the head of a patient can be stabilized.

B. Exemplary Sensor Assembly

Referring to FIG. 1, head stabilization system (10) comprises a sensor assembly including one or more sensors (102, 104) positioned on one or more components of skull clamp (20), such as one or more first pin holder assembly sensors (102) positioned on first pin holder assembly (40), and/or one or more second pin holder assembly sensors (104) positioned on second pin holder assembly (50). Sensors (102, 104) can be configured to detect one or more characteristics of one or more components of skull clamp (20) (e.g., position, displacement, orientation, load, force, etc.). For instance, sensors (102, 104) can include a position sensor (e.g., a linear variable differential transducer (LVDT), a piezo-electric transducer, a linear encoder, a rotary encoder, an optical sensor, etc.) for detecting an absolute position or location of one or more components of skull clamp (20) and/or a relative position or displacement of one or more components of skull clamp (20) relative to each other and/or a patient in terms of linear travel, rotational angle, and/or three-dimensional space. Sensors (102, 104) can include a force sensor (e.g., a strain gauge, a piezoresistive strain gauge, a capacitive sensor, an optical sensor, etc.) for detecting a force and/or load on one or more components of skull clamp (20). Still other suitable configurations for sensors (102, 104) will be discussed in more detail below.

Referring to FIGS. 2-3, first pin holder assembly sensors (102) include a first sensor (102a) positioned on wheel (44), a second sensor (102b) positioned on elongated member (48), and/or a fourth sensor (102c) positioned on pin tip (64). Sensors (102a, 102b, 102c) can thereby be configured to detect a linear position and/or rotational orientation of wheel (44), elongated member (48) and/or pin tip (64) respectively. For instance, sensors (102a, 102b, 102c) can determine a displacement of wheel (44), elongated member (48) and/or pin tip (64) relative to each other, relative to an adjustment path travelled by the component such as along axis (A), and/or relative to a patient.

In some versions, sensors (102a, 102b, 102c) detect a force or load exerted on a respective component of first pin holder assembly (40). For instance, sensor (102c) can detect a linear force at pin (60) along longitudinal axis (A) such that a low pin force can indicate slipping or slipping potential of pin (60) and/or a high pin force can indicate overtightening of pin holder assembly (40). In some versions, pin force can indicate a bone penetration depth of pin (60) within a skull of a patient.

First pin holder assembly sensors (102) can also include a fourth sensor (102d) positioned between torque screw (42) and bore of upright portion (26) and/or a fifth sensor (102e) positioned between pin (60) and elongate member (48). Sensors (102d, 102e) are configured to detect a shear force of torque screw (42) and/or pin (60) transverse to longitudinal axis (A), which also indicates slipping or slipping potential of pin (60). In some other versions, pin force can provide an indication of a contact angle of pin (60) relative to a skull of a patient such that a low and/or high contact angle can indicate slipping or slipping potential of pin (60). In this example, an optimal contact angle would be with a longitudinal axis of pin (60) being orthogonal with a tangent to the patient's skull. A less than optimal low and/or high contact angle would be ones that deviate from the ninety degree or orthogonal orientation by an amount that exceeds a threshold value for instance.

First pin holder assembly sensors (102) can also include a sixth sensor (102f) positioned between elongate member (48) and housing (62) of pin (60). Sensor (102f) is configured as a ring-shaped sensor that detects a linear or axial pinning force being applied to the patient along longitudinal axis (A). Based on data correlating bone penetration with pinning force, the data provided by sensor (102f) provides an indication of pin penetration within the skull of the patient. Based on the information collected by first pin holder assembly sensors (102), the operator and/or manufacture will have insight into what is happening at the interface of pin (60) with the bone of the patient. Various ways this information may be collected, analyzed, displayed, and used will be described in further detail below.

Referring to FIGS. 4-5, second pin holder assembly sensors (104) include first sensors (104a) positioned on each pin tip (64). Sensors (104a) can thereby be configured to detect a linear position and/or angular orientation of pin tips (64). For instance, sensors (104a) can determine a displacement pin tips (64) relative to each other, relative to an adjustment path travelled by the component such as along or about axes (B, C), and/or relative to a patient. For example, sensors (104a) can provide information and data to determine a relative position with respect to pin tip (64) of first pin holder assembly (40).

In some versions, sensors (104a) can detect a force or load exerted on a respective component of second pin holder assembly (50). For instance, sensor (104a) can detect a linear force at pin (60) of second pin holder assembly (50) along a longitudinal axis (B), or a longitudinal axis defined by pin (60) itself, such that a low pin force can indicate slipping or potential slippage of pin (60) based on inadequate bone penetration, and/or a high pin force can indicate potential over-penetration of pin (60) that may cause tissue or bone trauma. Second pin holder assembly sensors (104) can also include third sensors (104b) positioned between housing (62) of pins (60) and the bore or receptacle of rocker arm (54) receiving pins (60) to detect shear force of pins (60) transverse to longitudinal axis (B) or the longitudinal axes defined by pins (60) themselves, which also can indicate slipping or potential slipping of pins (60). In some other versions, pin force can indicate a contact angle of pins (60) relative to a skull of a patient such that a low and/or high contact angle can indicate slipping or potential slipping of one or both pins (60).

Second pin holder assembly sensors (104) can also include third sensors (104c) positioned between the bores of rocker arm (54) that receive pins (60) and housings (62) of pins (60) such that sensors (104c) are adjacent to housing (62) in a contacting fashion. Sensors (104c) are configured as a ring-shaped sensor that detect a linear or axial pinning force being applied to the patient along the longitudinal axes defined by respective pins (60) of second pin holder assembly (50). Based on data correlating bone penetration with pinning force, the data provided by sensors (104c) provides an indication of pin penetration within the skull of the patient. Based on the information collected by second pin holder assembly sensors (104), the operator and/or manufacture will have insight into what is happening at the interface of pins (60) with the bone of the patient. Various ways this information may be collected, analyzed, displayed, and used will be described in further detail below. In view of the teachings herein, other suitable configurations for sensors (102, 104) will be apparent to those of ordinary skill in the art.

C. Exemplary Connection Assembly

Referring back to FIG. 1, head stabilization system (10) comprises a connection assembly comprising a data processing unit (112) connected with sensors (102, 104) such that data collected by sensors (102, 104) is transmittable to data processing unit (112) for processing. Data processing unit (112) is further configured to connect with a power source (110) to provide power to data processing unit (112) and/or sensors (102, 104). Power source (110) can be wired to an electrical outlet or power supply and/or power source (110) can include batteries (e.g., disposable, rechargeable, etc.). In some versions, data processing unit (112) is configured to provide power to sensors (102, 104) such that separate power source (110) can be omitted. In some other versions, sensors (102, 104) may be powered separate from data processing unit (112) and/or power source (110), and instead be powered by separate batteries associated with sensors (102, 104). In view of the teachings herein, other ways to provide power to sensors (102, 104) and date processing unit (112) will be apparent to those of ordinary skill in the art.

Data processing unit (112) can be configured as a computing device comprising a processor, a memory or storage containing an operating system and other computer-readable instructions, and one or more communications features. For instance, the processor is operable to read and execute computer-readable instructions, which may be stored locally on data processing unit (112), or remote from data processing unit (112) yet accessible via one or more communications features. Exemplary computer-readable instructions that are executable by the processor, are described further below with respect to the description of the use of system (10) and can include one or more commands pertaining to how system (10) is operated and how resultant data from sensors (102, 104) is captured and processed.

In at least some instances, the memory is configured to store one or more applications, which represent, among other things, related computer-readable instructions for execution by the processor. One or more of communications features of data processing unit (112) are configured to transmit and receive data or other computer-readable and executable information. Communications features can include wired networking features or wireless networking features. The wireless networking features can include a Wi-Fi adapter, near field communication (NFC) features, and Bluetooth features. The components mentioned above for data processing unit (112) are not exhaustive and in view of the teachings herein, other features incorporable into data processing unit (112) will be apparent to those of ordinary skill in the art.

Accordingly, data processing unit (112) is configured to process data received by sensors (102, 104). For instance, data processing unit (112) can determine whether data received by sensors (102, 104) is below, equal to, and/or above a predetermined value (e.g., position, displacement, orientation, load, force, etc.). If data received by sensors (102, 104) is below, equal to, and/or below the predetermined value, data processing unit (112) can be configured to determine whether this indicates that stabilization of a patient in skull clamp (20) is not stable, that skull clamp (20) has been overtightened, and/or that a failure of a component or of a setup of skull clamp (20) has occurred. Such information can be provided to a user and/or a manufacturer of system (10).

In the illustrated version, data processing unit (112) is connected with a data interface (114) such that data analyzed by data processing unit (112) is transmittable to data interface (114) for display and/or further processing. Data interface (114) can be connected with data processing unit (112) by wired networking features and/or wireless networking features. Data interface (114) can include a display configured to present information to a user visually. Data interface (114) can further be configured to provide a visual and/or auditory alarm when data processing unit (112) determines that data received by sensors (102, 104) is below, equal to, and/or above the predetermined value. Data interface (114) can operate to provide feedback based on sensors (102, 104) in real time and/or from data stored in data processing unit (112). In some versions, data processing unit (112) includes a display that is configured to present information such that a separate data interface (114) can be omitted.

In some other versions, head fixation device (20) is fitted with one or more displays or indication features that are configured and operable to indicate to an operator state of the one or more detected characteristics of head fixation device (20). By way of example only, and not limitation, head fixation device (20) in some versions includes indication features (70) that may have the form of any one or more of light features, audible features, etc. to communicate the status of the one or more characteristics of head fixation device (20) detected by sensors (102, 104). For instance, indication feature (70) may present as a solid green LED when the one or more characteristics, e.g., pinning force, is within an acceptable deviation from a prescribed target. However, indication feature (70) may present as a flashing red LED when the same one or more characteristics are outside of the acceptable deviation from the prescribed target. In view of the teachings herein, other ways to communicate the status of the detected one or more characteristics of head fixation device (20) will be apparent to those of ordinary skill in the art.

In the illustrated version, data interface (114) is further configured to connect data processing unit (112) with other devices, such as a navigation system (116) and an augmented reality system (118). Data interface (114) is thereby configured to transmit data received from data processing unit (112) to navigation system (116) and/or augmented reality system (118). Similarly, data from navigation system (116) and/or augmented reality system (118) can be transmitted to data interface (114) and/or data processing unit (112) such that data from sensors (102, 104) can be combined with data from navigation system (116) and/or augmented reality system (118).

Data interface (114) can be connected with navigation system (116) and/or augmented reality system (118) by wired networking features and/or wireless networking features. Navigation system (116) can be configured to display a visual map or diagram of a patient's head for the medical procedure. Accordingly, navigation system (116) can include data received by navigation system (116) from data interface (114) to display one or more components of skull clamp (20) relative to the map or diagram of the patient's head for the medical procedure. Augmented reality system (118) can be configured to display a visual image of the patient's skull. Accordingly, augmented reality system (118) can include data received by augmented reality system (118) from data interface (114) to display one or more components of skull clamp (20) relative to the image. Navigation system (116) and/or augmented reality system (118) can thereby show the relation of pins (60) of skull clamp (20) relative to a patient's skull. In some versions, navigation system (116) and/or augmented reality system (118) can be omitted or combined.

D. Exemplary Method of Use

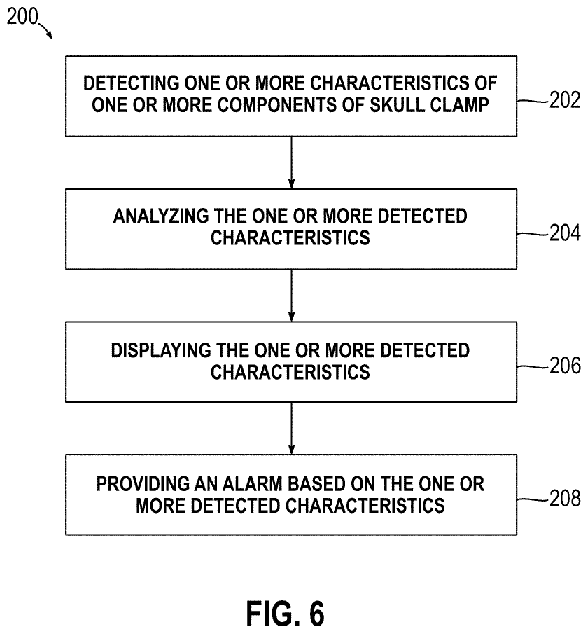

FIG. 6 shows an exemplary method (200) for operating a head stabilization system (10). Method (200) comprises detecting one or more characteristics of one or more components of skull clamp (20) (step (202)), analyzing the one or more detected characteristics (step (204)), displaying the one or more detected characteristics (step (206)), and/or providing an alarm based on the one or more detected characteristics (step (208)). For instance, sensors (102, 104) can detect one or more characteristics (e.g., position, displacement, orientation, load, force, etc.) of pin holder assemblies (40, 50) of skull clamp (20). Data processing unit (112) can then receive data measured or detected by sensors (102, 104) such that data processing unit (112) can analyze the one or more characteristics detected by sensors (102, 104). For instance, data processing unit (112) can process the data received by sensors (102, 104) to compare the detected characteristic to a predetermined value to determine whether the detected characteristic is above, equal to, and/or below the predetermined value. Based on the comparison of the detected characteristic with the predetermined value, data processing unit (112) can provide a visual and/or auditory alarm to indicate whether the detected characteristic has deviated from and/or exceeds the predetermined value. Data processing unit (112) can further display the detected characteristic via data processing unit, data interface (114), navigation system (116), and/or augmented reality system (118). A user can thereby adjust pin holder assemblies (40, 50) of skull clamp (20) based on the feedback provided by head stabilization system (10).

For illustrative purposes only, sensors (102, 104) can measure a force or load being exerted on pins (60) during a medical procedure. Data processing unit (112) can then receive and process the data measured by sensors (102, 104) to determine how the force detected at sensors (102, 104) compares with a predetermined value. For instance, a force above the predetermined value can indicate that pins (60) have been overtightened. In some versions, a force below the predetermined value can indicate that pins (60) have slipped or lessened penetration with the bone. Accordingly, data processing unit (112) can provide an alarm to indicate that a force at pins (60) is outside of a desired range based on the comparison of the detected force and the predetermined value. A user can adjust the position of pin holder assemblies (40, 50) of skull clamp (20) based on the feedback of head stabilization system (10) to increase or reduce force at pins (60) depending on the comparison of the detected value to the predetermined, threshold, and/or target value. Data processing unit (112) can further display the detected force via data processing unit, data interface (114), navigation system (116), and/or augmented reality system (118). Accordingly, a user can also adjust the pin holder assemblies (40, 50) of skull clamp (20) based on the display of head stabilization system (10) in these other components or systems. Still other suitable methods for operating head stabilization system (10) will be apparent to one with ordinary skill in the art in view of the teachings herein.

II. Alternate Exemplary Head Stabilization System

An exemplary head stabilization system comprises a sensor assembly and a connection assembly connected with a head fixation device. The sensor assembly comprises one or more sensors positioned on one or more components of the head fixation device and is configured to detect one or more characteristics of the head fixation device. The connection assembly comprises a data processing unit that is configured to receive and process data detected by the sensor assembly. For instance, the data processing unit can determine whether data received from the sensor assembly has reached and/or exceeded a predetermined or threshold value, which can indicate a condition or state that may require corrective action or intervention. The connection system can then be configured to communicate feedback to a user and/or manufacturer based on the detected characteristic. The head fixation device can be adjusted based on the feedback to avoid possible problems during a medical procedure

A. Alternate Exemplary Head Fixation Device

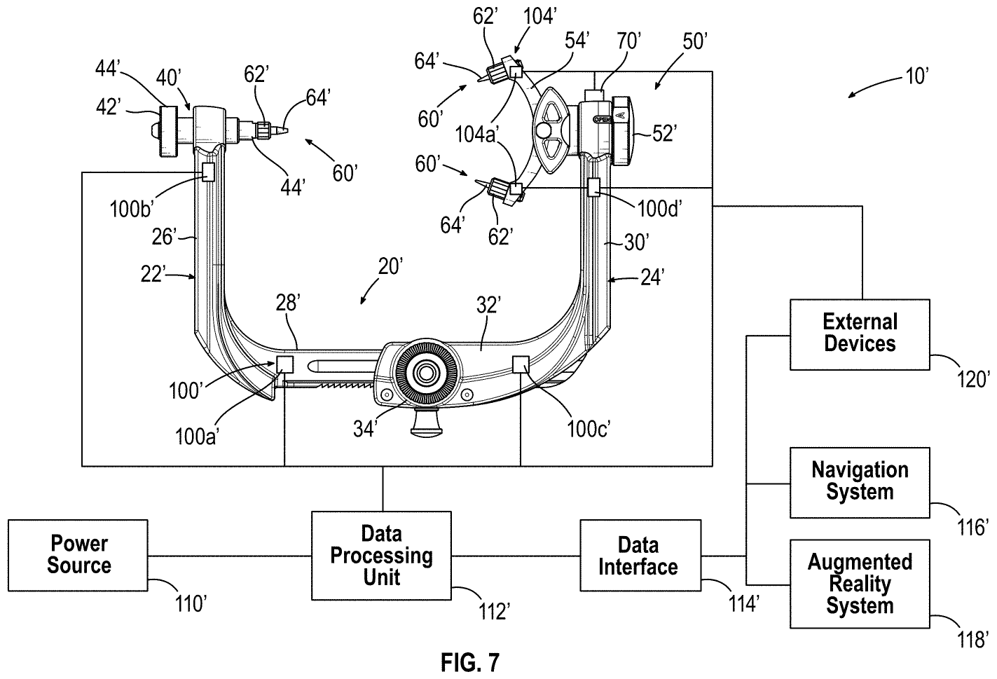

FIG. 7 illustrates an exemplary head stabilization system (10′) for an exemplary head stabilization or fixation device (20′). Throughout the specification the term “HFD” is used interchangeably with the terms “head stabilization device,” “head fixation device,” or “skull clamp.” In the illustrated versions here HFD (20′) has the shape or form of a skull clamp. While the present example illustrates the HFD as a U-shaped skull clamp, the teachings herein may be applied to other forms of HFDs as will be understood by those of ordinary skill in the art in view of the teachings herein. Skull clamp (20′) can be made from a composite material, a polymer material (e.g., polyether ether ketone (PEEK), polytetrafluoroethylene (PTFE), etc.), and/or a metal material (e.g., aluminum, stainless steel, titanium, etc.). Skull clamp (20′) comprises a first arm (22′) and a second arm (24′). First arm (22′) is connectable with second arm (24′) to form skull clamp (20′) having a U-shape. First arm (22′) comprises an upright portion (26′) and a lateral portion (28′). Similarly, second arm (24′) comprises an upright portion (20′) and a lateral portion (22′). Skull clamp (20′) is adjustable to accommodate a variety of head sizes by translating first arm (22′) relative to second arm (24′) or vice versa. Skull clamp (20′) is further connectable to other structures, such as a positioning adapter or a base unit that is further connectable with an operating table, etc., by way of an attachment interface (34′). As shown in the present example of FIG. 7, upright portion (26′) of first arm (22′) connects with a first pin holder assembly (40′), while upright portion (30′) of second arm (24′) connects with a second pin holder assembly (50′).

First pin holder assembly (40′) comprises a torque screw (42′) configured to adjust an amount of clamping force skull clamp (20′) applies to the head of the patient. Torque screw (42′) extends through a bore in upright portion (26′) of arm (22′). Torque screw (42′) comprises an actuator in the form of a wheel (44′). As wheel (44′) is rotated, a pinning force applied by torque screw (42′) increases or decreases depending on the direction of rotation of wheel (44′). In view of the teaching herein, other ways to modify or use torque screw (42′), or another similar structure, to control an amount of pinning force applied will be apparent to those of ordinary skill in the art.

At its distal end, torque screw (42′) selectively retains a pin (60′), which includes a housing (62′) and a pin tip (64′). In use, at least a distal tip of pin (60′) contacts the head of the patient. When wheel (44′) is rotated in a first direction, a spring within torque screw (42′) is compressed and thereby exerts an increased force on pin (60′) in a direction toward the patient's head. Force applied by pin (60′) to the head of the patient can be decreased by rotation of wheel (44′) in the opposite direction. Accordingly, as wheel (44′) is rotated, a pinning force applied by torque screw (42′) and/or pin (60′) increases or decreases depending on the direction of rotation of wheel (44′).

Second pin holder assembly (50′) connects with upright portion (30′) of arm (24′) as shown. Second pin holder assembly (50′) comprises rocker arm (54′) that selectively retains a pair of pins (60′), which each include housing (62′) and pin tip (64′). Pin holder assembly (50′) is rotatably adjustable its longitudinal axis extending through a bore in upright portion (30′). This rotation can be selectively controlled such that in some versions the rotatable position of pin holder assembly (50′) can be locked in position or unlocked for adjustment. In some other versions, pin holder assembly (50′) is rotatably adjustable about its longitudinal axis and is secured in its rotational position based on the force applied to the patient's head when clamping. In view of the teachings herein, various ways to configure pin holder assembly (50′) to provide locking and unlocking states for rotational adjustment will be apparent to those of ordinary skill in the art.

Second pin holder assembly (50′) is also configured such that rocker arm (54′) is pivotably adjustable. Regardless of the rotational or pivotal adjustments, the force applied to the head of the patient via torque screw (42′) of first pin holder assembly (40′) as described above, causes force to also be applied to the head of the patient from second pin holder assembly (50′) and its pins (60′) that are configured to contact the head of the patient. With the configuration described above for skull clamp (20′), the head of a patient can be stabilized.

B. Alternate Exemplary Sensor Assembly

Referring to FIG. 7, head stabilization system (10′) comprises a sensor assembly including one or more sensors positioned on one or more components of skull clamp (20′), such as one or more frame sensors (100′) positioned on one or more of arms (22′, 24′), and/or one or more second pin holder assembly sensors (104′) positioned on second pin holder assembly (50′). In some versions, one or more sensors can be provided on first pin holder assembly (40′) instead or in addition to the sensors described above.

Sensors (100′, 104′) can be configured to detect one or more characteristics of one or more components of skull clamp (20′) (e.g., position, displacement, orientation, vibration, exposure, temperature, etc.). For instance, sensors (100′, 104′) can include a position sensor (e.g., a linear variable differential transducer (LVDT), a piezo-electric transducer, a linear encoder, a rotary encoder, an optical sensor, etc.) for detecting an absolute position or location of one or more components of skull clamp (20′) and/or a relative position or displacement of one or more components of skull clamp (20′) relative to each other and/or a patient in terms of linear travel, rotational angle, and/or three-dimensional space. Sensors (100′, 104′) can include a vibration or shock sensor for detecting an impact on one or more components of skull clamp (20′). Sensors (100′, 104′) can include a temperature sensor (e.g., a thermocouple, a resistance temperature detector (RTD), a thermistor, etc.) for detecting a temperature at one or more components of skull clamp (20′). Sensors (100′, 104′) can include an exposure sensor for detecting the presence of one or more substances in proximity to one or more components of skull clamp (20′). Still other suitable configurations for sensors (100′, 104′) will be discussed in more detail below.

In the illustrated version, frame sensor(s) (100′) include a first sensor (100a′) positioned on lateral portion (28′) of first arm (22′), a second sensor (100b′) positioned on upright portion (26′) of first arm (22′), a third sensor (100c′) positioned on lateral portion (32′) of second arm (24′), and/or a fourth sensor (100d′) positioned on upright portion (30′) of second arm (24′). Frame sensors (100′) can thereby be configured to detect the position or adjustment of first arm (22′) relative to second arm (24′) when first arm (22′) is translated relative to second arm (24′) or vice versa. Frame sensors (100′) can further be configured to detect a rotational orientation of first arm (22′) and/or second arm (24′) such as relative to an adapter or operating table. In some other versions, frame sensors (100′) are configured to detect a position of first arm (22′) and/or second arm (24′) relative to a head of a patient being stabilized within skull clamp (20′).

Second pin holder assembly sensors (104′) include a sensors (104a′) positioned on rocker arm (54′) as shown. Sensors (104a′) can thereby be configured to detect a linear position and/or rotational orientation of rocker arm (54′). For instance, sensors (104a′) can determine a displacement of rocker arm (54′) relative to an adjustment path travelled by the component, and/or relative to a patient. Still other suitable configurations for sensors (100′, 104′) will be apparent to one with ordinary skill in the art in view of the teachings herein.

C. Alternate Exemplary Connection Assembly

Still referring to FIG. 7, head stabilization system (10′) comprises a connection assembly comprising a data processing unit (112′) connected with sensors (100′, 104′) such that data collected by sensors (100′, 104′) is transmittable to data processing unit (112′) for processing. Data processing unit (112′) is further configured to connect with a power source (110′) to provide power to data processing unit (112′) and/or sensors (100′, 104′). Power source (110′) can be wired to an electrical outlet or power supply and/or power source (110′) can include batteries (e.g., disposable, rechargeable, etc.). In some versions, data processing unit (112′) is configured to provide power to sensors (100′, 104′) such that separate power source (110′) can be omitted. In some other versions, sensors (100′, 104′) may be powered separate from data processing unit (112′) and/or power source (110′), and instead be powered by separate batteries associated with sensors (100′, 104′). In view of the teachings herein, other ways to provide power to sensors (100′, 104′) and date processing unit (112′) will be apparent to those of ordinary skill in the art.

Data processing unit (112′) can be configured as a computing device comprising a processor, a memory or storage containing an operating system and other computer-readable instructions, and one or more communications features. For instance, the processor is operable to read and execute computer-readable instructions, which may be stored locally on data processing unit (112′), or remote from data processing unit (112′) yet accessible via one or more communications features. Exemplary computer-readable instructions that are executable by the processor, are described further below with respect to the description of the use of system (10′) and can include one or more commands pertaining to how system (10′) is operated and how resultant data from sensors (100′, 104′) is captured and processed.

In at least some instances, the memory is configured to store one or more applications, which represent, among other things, related computer-readable instructions for execution by the processor. One or more of communications features of data processing unit (112′) are configured to transmit and receive data or other computer-readable and executable information. Communications features can include wired networking features or wireless networking features. The wireless networking features can include a Wi-Fi adapter, near field communication (NFC) features, and Bluetooth features. The components mentioned above for data processing unit (112′) are not exhaustive and in view of the teachings herein, other features incorporable into data processing unit (112′) will be apparent to those of ordinary skill in the art.

Accordingly, data processing unit (112′) is configured to process data received by sensors (100′, 104′). For instance, data processing unit (112′) can determine whether data received by sensors (100′, 104′) is below, equal to, and/or above a predetermined value (e.g., position, displacement, orientation, vibration, exposure, temperature, etc.). If data received by sensors (100′, 104′) is above, equal to, and/or below the predetermined value, data processing unit (112′) can be configured to determine whether this indicates an undesirable condition or status of one or more components of skull clamp (20′) requiring corrective action or intervention. Such information can be provided to a user and/or a manufacturer of system (10′).

In the illustrated version, data processing unit (112′) is connected with a data interface (114′) such that data analyzed by data processing unit (112′) is transmittable to data interface (114′) for display and/or further processing. Data interface (114′) can be connected with data processing unit (112′) by wired networking features and/or wireless networking features. Data interface (114′) can include a display configured to present information to a user visually. Data interface (114′) can further be configured to provide a visual and/or auditory alarm when data processing unit (112′) determines that data received by sensors (100′, 104′) is below, equal to, and/or above the predetermined value. Data interface (114′) can operate to provide feedback based on sensors (100′, 104′) in real time and/or from data stored in data processing unit (112′). In some versions, data processing unit (112′) includes a display that is configured to present information such that a separate data interface (114′) can be omitted.

In some other versions, head fixation device (20′) is fitted with one or more displays or indication features that are configured and operable to indicate to an operator state of the one or more detected characteristics of head fixation device (20′). By way of example only, and not limitation, head fixation device (20′) in some versions includes indication features (70′) that may have the form of any one or more of light features, audible features, etc. to communicate the status of the one or more characteristics of head fixation device (20′) detected by sensors (100′, 104′). For instance, indication feature (70′) may present as a solid green LED when the one or more characteristics, e.g., vibration, is within an acceptable deviation from a prescribed target. However, indication feature (70′) may present as a flashing red LED when the same one or more characteristics are outside of the acceptable deviation from the prescribed target. In view of the teachings herein, other ways to communicate the status of the detected one or more characteristics of head fixation device (20′) will be apparent to those of ordinary skill in the art.

In the illustrated version, data interface (114′) is further configured to connect data processing unit (112′) with other devices, such as a navigation system (116′) and an augmented reality system (118′). Data interface (114′) is thereby configured to transmit data received from data processing unit (112′) to navigation system (116′) and/or augmented reality system (118′). Data interface (114′) can be connected with navigation system (116′) and/or augmented reality system (118′) by wired networking features and/or wireless networking features. Navigation system (116′) can be configured to display a visual map or diagram of a patient's brain for the medical procedure. Accordingly, navigation system (116′) can include data received by navigation system (116′) from data interface (114′) to display one or more components of skull clamp (20′) relative to the map or diagram of the patient's brain for the medical procedure. Augmented reality system (118′) can be configured to display a visual image of the patient's skull. Accordingly, augmented reality system (118′) can include data received by augmented reality system (118′) from data interface (114′) to display one or more components of skull clamp (20′) relative to the image. Navigation system (116′) and/or augmented reality system (118′) can thereby show the relation of pins (60′) or other components of skull clamp (20′) relative to a patient's skull and/or the surrounding environment. In some versions, navigation system (116′) and/or augmented reality system (118′) can be omitted.

D. Alternate Exemplary Method of Use

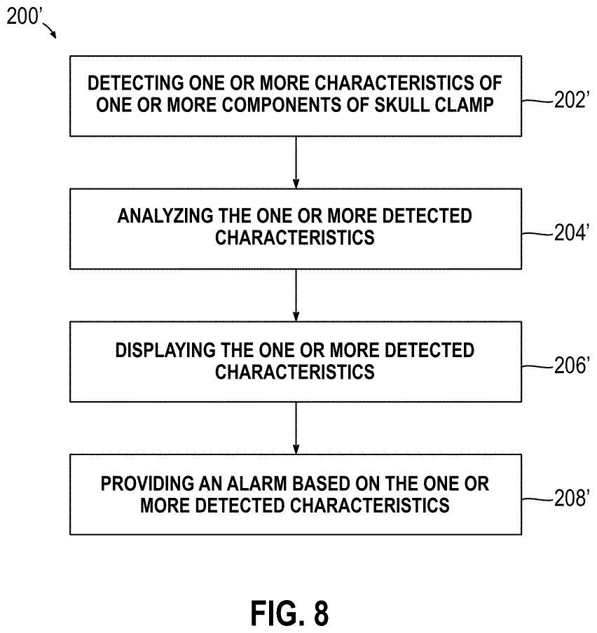

FIG. 8 shows an exemplary method (200′) for operating head stabilization system (10′). Method (200′) comprises detecting one or more characteristics of one or more components of skull clamp (20′) (step (202′)), analyzing the one or more detected characteristics (step (204′)), displaying the one or more detected characteristics (step (206′)), and/or providing an alarm based on the one or more detected characteristics (step (208′)). For instance, sensors (100′, 104′) can detect one or more characteristics, as mentioned above, of arms (22′, 24′) and/or pin holder assembly (50′) of skull clamp (20′). Data processing unit (112′) can then receive data measured or detected by sensors (100′, 104′) such that data processing unit (112′) can analyze the one or more characteristics detected by sensors (100′, 104′). For instance, data processing unit (112′) can process the data received by sensors (100′, 104′) to compare the detected characteristic to a predetermined value to determine whether the detected characteristic is above, equal to, and/or below the predetermined value. Based on the comparison of the detected characteristic with the predetermined value, data processing unit (112′) can provide a visual and/or auditory alarm to indicate whether the detected characteristic has deviated from and/or exceeds the predetermined value. Data processing unit (112′) can further display the detected characteristic via data processing unit, data interface (114′), navigation system (116′), and/or augmented reality system (118′). A user can thereby take corrective or intervention action based on the feedback provided by head stabilization system (10′). The below sections describe specific exemplary detection features and methods. Furthermore, additional exemplary detection features and methods will be apparent to those of ordinary skill in the art in view of the teachings herein.

E. Exemplary Reprocessing and/or Sterilization Detection

In some versions, one or more components of skull clamp (20′) can be subjected to higher temperatures to sterilize and/or reprocess the one or more components such that the one or more components can be reused in another medical procedure. For instance, in some versions reprocessing may expose the components to temperatures about or exceeding 90 degrees Celsius for a prescribed duration; and in some versions sterilization may expose the components to temperature about or exceeding 120 degrees Celsius for a prescribed duration. In some instances, it can be desirable to determine whether one or more components of skull clamp (20′) has been reprocessed and/or sterilized and/or how many times the one or more components have been reprocessed and/or sterilized. Accordingly, sensors (100′, 104′) can include a temperature sensor for detecting a temperature at or near sensors (100′, 104′). Such temperature data detected by sensors (100′, 104′) can be transmitted to data processing unit (112′). Data processing unit (112′) can process and analyze the sensor data by comparing the detected data with a predetermined temperature value to determine whether the one or more components of skull clamp (20′) has been exposed to reprocessing temperatures and/or sterilization temperatures. For instance, if the detected data exceeds the predetermined temperature value, data processing unit (112′) can determine that the one or more components have been subjected to reprocessing and/or sterilization. Data processing unit (112′) can also determine how many times the detected data exceeds the predetermined temperature value to determine the number of times the one or more components have been reprocessed and/or sterilized. In some versions, data processing unit (112′) can provide an alert to indicate when one or more components of skull clamp (20′) has exceeded a desired amount of reprocessing and/or sterilization. Still other suitable configurations and/or methods for providing reprocessing and/or sterilization detection by head stabilization system (10′) will be apparent to one with ordinary skill in the art in view of the teachings herein.

F. Exemplary Steam Sterilization Detection

In some versions, one or more components of skull clamp (20′) can be subjected to steam to sterilize the one or more components such that the one or more components can be reused in another medical procedure. In some instances, it can be desirable to determine whether one or more components of skull clamp (20′) has been exposed to steam. Accordingly, sensors (100′, 104′) can include a sensor for detecting the presence of steam at or near sensors (100′, 104′). Such data detected by sensors (100′, 104′) can be transmitted to data processing unit (112′). Data processing unit (112′) can process and analyze the sensor data to determine whether the one or more components of skull clamp (20′) has been exposed to steam. Data processing unit (112′) can also determine how many times the one or more components have been subjected to steam. In some versions, data processing unit (112′) can provide an alert to indicate when or how many times the one or more components of skull clamp (20′) has been exposed to steam. Still other suitable configurations and/or methods for providing steam sterilization detection by head stabilization system (10′) will be apparent to one with ordinary skill in the art in view of the teachings herein.

G. Exemplary Reuse Detection

In some versions, one or more components of skull clamp (20′) can be reused in another medical procedure. In some instances, it can be desirable to determine whether one or more components of skull clamp (20′) have previously been used, the duration of the previous use and/or whether the one or more components needs to be replaced. Accordingly, one or more components of arms (22′, 24′), pin holder assemblies (40′, 50′), and/or sensors (100′, 104′) can include a unique identifier (e.g., radio-frequency identification (RFID) chip). Data processing unit (112′) can be configured to connect with and/or detect the identifier such as by wireless networking features (e.g., Wi-Fi adapter, near field communication (NFC) features, Bluetooth features, etc.). Data processing unit (112′) can thereby be configured to determine whether the one or more components have previously been used, the duration of the previous use and/or whether the one or more components needs to be replaced based on the detection of the identifier. For instance, data processing unit (112′) can determine whether the amount of time that data processing unit (112′) is connected with the identifier has exceeded a predetermined duration value for the one or more components. If the predetermined duration value has been exceeded, data processing unit (112′) can indicate or provide an alarm to replace the one or more components. In some versions, data processing unit (112′) can determine whether the one or more components have been replaced with a previously used component when data processing unit (112′) is reconnected with an identifier that data processing unit (112′) has been previously connected. If data processing unit (112′) is reconnected with a previously used identifier, data processing unit can indicate or provide an alarm that the one or more components have not been replaced. Still other suitable configurations and/or methods for providing reuse detection by head stabilization system (10′) will be apparent to one with ordinary skill in the art in view of the teachings herein.

H. Exemplary Shock Detection

In some instances, it can be desirable to determine whether one or more components of skull clamp (20′) have been subjected to shock or physical impact that could damage the one or more components, such as during shipment and/or handling of the one or more components. Accordingly, sensors (100′, 104′) can include a one or more shock sensors to detect vibration and/or a physical impact of the one or more components. For instance, sensors (100′, 104′) can include an accelerometer to detect an acceleration of a component such when a component is dropped, a harmonic oscillator that can be displaced from an equilibrium position during an impact, a brittle component with a known fragility that can break during an impact, and/or other suitable shock sensors configured to detect a shock or physical impact. Data processing unit (112′) can be configured to connect with and/or detect such shock sensors to determine whether the one or more components have previously been subjected to a shock or physical impact. If data processing unit (112′) determines that a shock has occurred, data processing unit can indicate or provide an alarm that the one or more components have experienced a shock. Still other suitable configurations and/or methods for providing shock detection by head stabilization system (10′) will be apparent to one with ordinary skill in the art in view of the teachings herein.

I. Exemplary Sensor Communication to External Devices

In some versions, sensors (100′, 102′) are configured such that they are able to communicate their location to external devices (120′), as shown in FIG. 7. Furthermore, these sensors (100′, 102′) may be configured to communicate not only their location in space, but also the precise location of all of head fixation device (20′). In this way, external devices (120′) are provided with spatial location information for head fixation device (20′) and can use this information during the procedure as necessary to avoid complications and achieve desired outcomes.

By way of example only, and not limitation, external device (120′) represents a robotic surgical device that may or may not be assisted by a surgeon. In such an example, sensors (100′, 104′) communicate their location information and data concerning head fixation device (20′) to the robotic surgical device. This communication may occur via data processing unit (112′) and data interface (114′) in some versions. In other versions, this communication may occur directly between sensors (100′, 104′) and external devices (120′), e.g., using a wireless communication modality. Still in some other versions, sensors (100′, 104′) may instead, or in addition, be detectable by external devices (120′) via wireless communication modalities such as Bluetooth, RFID, NFC, etc. With the robotic surgical device of the present example having the information and data on the location of head fixation device (20′), the robotic surgical device can effectively “see” head fixation device (20′) and navigate around head fixation device (20′) during the procedure being carried out.

In still another example, external device (120′) represents a scanner that collects image scans pre, intra, or post procedure. In a similar fashion as described above, sensors (100′, 104′) communicate their spatial location and that of head fixation device (20′) to the scanner. Accordingly, when conducting the scans, the scanner has the information necessary to navigate around head fixation device (20′) to achieve a desired outcome for the scan being acquired. In some examples where sensors (100′, 104′) are detected by external devices (120′), sensors (100′, 104′) can include optical features, RFID features, NFC features, etc.; however, other suitable sensor types may be used as well and will be apparent to those of ordinary skill in the art in view of the teachings herein.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A device for stabilizing a patient comprising a head fixation device comprising a first arm coupled with a first pin holder assembly and a second arm coupled with a second pin holder assembly, and one or more sensors positioned on the head fixation device, wherein the one or more sensors are configured to detect one or more characteristics of the head fixation device.

### EXAMPLE 2

The device of Example 1, further comprising a data processing unit connected with the one or more sensors such that the data processing unit is configured to receive data from the one or more sensors, wherein the data processing unit is configured to analyze the characteristic detected by the one or more sensors to provide feedback based on the detected characteristic.

### EXAMPLE 3

The device of any one or more of Example 1 through Example 2, wherein the one or more characteristics of the head fixation device includes one or more of a position, displacement, orientation, load, and force of the head fixation device.

### EXAMPLE 4

The device of any one or more of Example 1 through Example 3, wherein the first pin holder assembly comprises a torque screw assembly including an actuator and a pin configured to contact a head, wherein the actuator is configured to adjust the pin relative to the head.

### EXAMPLE 5

The device of Example 4, wherein at least one sensor is positioned on the actuator configured to detect one or more of a position, displacement, and angular orientation of the actuator.

### EXAMPLE 6

The device of any one or more of Example 4 through Example 5, wherein at least the one sensor is positioned adjacent the pin in a contacting configuration.

### EXAMPLE 7

The device of Example 4 through Example 6, wherein the at least one sensor is configured to detect one or more of a position, displacement, angular orientation, and force of the pin.

### EXAMPLE 8

The device of any one or more of Example 1 through Example 7, wherein the second pin assembly comprises a rocker arm and a pin coupled with the rocker arm.

### EXAMPLE 9

The device of Example 8, wherein at least one sensor is positioned on the pin of the second pin assembly and the sensor is configured to detect one or more of a position, displacement, angular orientation, and force of the pin.

### EXAMPLE 10

The device of any one or more of Example of 2 through Example 9, further comprising a power source connected with the one or more sensors and the data processing unit, wherein the power source is configured to provide power to the one or more sensors and the data processing unit.

### EXAMPLE 11

The device of any one or more of Example 2 through Example 10, wherein the data processing unit is configured to determine whether the fixation provided by the head fixation device is stable.

### EXAMPLE 12

The device of any one or more of Example 2 through Example 11, wherein the data processing unit is configured to compare the characteristic detected by the one or more sensors with a predetermined value to determine whether the fixation provided by the head fixation device is stable.

### EXAMPLE 13

The device of any one or more of Example 2 through Example 12, wherein the data processing unit is configured to provide an alarm to indicate when the fixation provided by the head fixation device is not stable.

### EXAMPLE 14

The device of any one or more of Example 2 through Example 13, further comprising a data interface connected with the data processing unit such that the characteristic analyzed by the data processing unit is transmittable to the data interface.

### EXAMPLE 15

The device of Example 14, wherein the data interface comprises a display for displaying the characteristic analyzed by the data processing unit.

### EXAMPLE 16

The device of any one or more of Example 14 through Example 15, wherein the data interface is configured to connect the data processing unit with remote devices.

### EXAMPLE 17

The device of any one or more of Example 14 through Example 16, wherein the data interface is configured to connect the data processing unit with a navigation system configured to display a diagram of the analyzed characteristic with a head of the patient.

### EXAMPLE 18

The device of any one or more of Example 14 through Example 17, wherein the data interface is configured to connect the data processing unit with an augmented reality system configured to display an image of the analyzed characteristic with a head of the patient.

### EXAMPLE 19

The device of Example 1, further comprising a connection assembly connected with the sensor assembly, wherein the connection assembly comprises a data processing unit configured to receive data from the one or more sensors, wherein the connection assembly is configured to analyze the characteristic detected by the one or more sensors to provide feedback of the detected characteristic.

### EXAMPLE 20

A method of operating a head stabilization system with a head fixation device having a first arm coupled with a first pin holder assembly and a second arm coupled with a second pin holder assembly, one or more sensors positioned on the head fixation device, and a data processing unit connected with the one or more sensors such that the data processing unit is configured to receive data from the one or more sensors, comprises the steps of detecting one or more characteristics of the head fixation device, and analyzing the one or more detected characteristics.

### EXAMPLE 21

The method of Example 20 further comprising displaying the one or more detected characteristics.

### EXAMPLE 22

The method of any one or more of Example 20 through Example 21, further comprising providing an alarm based on the one or more detected characteristics.

### EXAMPLE 23

The method of any one or more of Example 20 through Example 22, wherein the one or more characteristics includes one or more of a position, displacement, orientation, load, and force of the head fixation device.

### EXAMPLE 24

The method of any one or more of Example 20 through Example 23, further comprising adjusting the head fixation device based on the one or more detected characteristics.

### EXAMPLE 25

A device for stabilizing a patient comprises a head fixation device with a first arm coupled with a first pin holder assembly and a second arm coupled with a second pin holder assembly, and one or more sensors positioned on the head fixation device, wherein the one or more sensors are configured to detect one or more characteristics of the head fixation device.

### EXAMPLE 26

The device of Example 25, further comprising a data processing unit connected with the one or more sensors such that the data processing unit is configured to receive data from the one or more sensors, wherein the data processing unit is configured to analyze the characteristic detected by the one or more sensors to provide feedback based on the detected characteristic.

### EXAMPLE 27

The device of any one or more of Example 25 through Example 26, wherein a first sensor of the one or more sensors is positioned on a select one of the first arm and the second arm of the head fixation device such that the first sensor is configured to detect a position of select one of the first arm and the second arm.

### EXAMPLE 28

The device of Example 27, wherein a second sensor of the one or more sensors is positioned on the other of the select one of the first arm and the second arm of the head fixation device such that the second sensor is configured to detect a position of the other of the select one of the first arm and the second arm, wherein the first arm is translatable relative to the second arm, wherein the first sensor and the second sensor are configured to detect the position of the first arm relative to the second arm.

### EXAMPLE 29

The device of any one or more of Example 25 through Example 28, wherein the one or more characteristics of the head fixation device includes one or more of a position, displacement, orientation, vibration, exposure, and temperature of the head fixation device.

### EXAMPLE 30

The device of any one or more of Example 25 through Example 29, wherein the second pin assembly comprises a rocker arm and a pin coupled with the rocker arm.

### EXAMPLE 31

The device of Example 30, wherein at least one sensor is positioned on the rocker arm configured to detect one or more of a position, displacement, and angular orientation of the rocker arm.

### EXAMPLE 32

The device of any one or more of Example 26 through Example 31, further comprising a power source connected with the one or more sensors and the data processing unit, wherein the power source is configured to provide power to the one or more sensors and the data processing unit.

### EXAMPLE 33

The device of any one or more of Example 26 through Example 33 further comprising a data interface connected with the data processing unit such that characteristic analyzed by the data processing unit is transmittable to the data interface.

### EXAMPLE 34

The device of Example 33, wherein the data interface comprises a display for displaying the characteristic analyzed by the data processing unit.

### EXAMPLE 35

The device of any one or more of Example 33 through Example 34, wherein the data interface is configured to connect the data processing unit with a select one or more of a navigation system, an augmented reality system, and/or an external device.

### EXAMPLE 36

The device of any one or more of Example 25 through Example 35, wherein the one or more sensors includes a temperature sensor configured to detect when the head fixation device is exposed to reprocessing temperatures.

### EXAMPLE 37

The device of any one or more of Example 25 through Example 36, wherein the data processing unit is configured to determine the number of instances the head fixation device has been reprocessed.

### EXAMPLE 38

The device of Example 37, wherein the data processing unit is configured to provide an alarm when the number of instances that the head fixation device has been reprocessed exceeds a predetermined value.

### EXAMPLE 39

The device of any one or more of Example 25 through Example 38, wherein the one or more sensors are configured to detect when the head fixation device is exposed to sterilization temperatures.

### EXAMPLE 40

The device of Example 39, wherein the data processing unit is configured to determine the number of instances the head fixation device has been exposed to sterilization temperatures.

### EXAMPLE 41

The device of Example 40, wherein the data processing unit is configured to provide an alarm when the number of instances that the head fixation device has been exposed to sterilization temperatures exceeds a predetermined value.

### EXAMPLE 42

The device of any one or more of Example 26 through Example 41, wherein the head fixation device includes a unique identifier, wherein the data processing unit is configured to detect the unique identifier.

### EXAMPLE 43

The device of Example 42, wherein the data processing unit is configured to determine when one or more components of the head fixation device needs to be replaced based on the unique identifier.

### EXAMPLE 44

The device of any one or more of Example 42 or Example 43, wherein the data processing unit is configured to determine whether the one or more components of the head fixation device has been replaced based on the unique identifier.

### EXAMPLE 45

The device of any one or more of Example 26 through Example 44, wherein the data processing unit is configured to provide an alarm when one or more components of the head fixation device needs to be replaced.

### EXAMPLE 46

The device of any one or more of Example 25 through Example 45, wherein the one or more sensors includes a shock sensor configured to detect when the head fixation device has been subjected to a physical impact.

### EXAMPLE 47

The device of Example 46, wherein the data processing unit is configured to determine whether the head fixation device has been subjected to a physical impact based on the data received by the shock sensor.

### EXAMPLE 48

The device of any one or more of Example 46 through Example 47, wherein the data processing unit is configured to provide an alarm when the head fixation device has been subjected to a physical impact.

### EXAMPLE 49

A device for stabilizing a patient comprises a head fixation device with a first arm coupled with a first pin holder assembly and a second arm coupled with a second pin holder assembly, and a sensor assembly comprising one or more sensors positioned on the head fixation device, wherein the one or more sensors are configured to communicate location information to an external device, wherein the location information comprises location information pertaining to the spatial location of the head fixation device relative to the external device.

### EXAMPLE 50

A method of using a head stabilization system having a head fixation device with a first arm coupled with a first pin holder assembly and a second arm coupled with a second pin holder assembly, one or more sensors positioned on the head fixation device, and a data processing unit connected with the one or more sensors such that the data processing unit is configured to receive data from the one or more sensors, the method comprises detecting one or more characteristics of the head fixation device, and analyzing the one or more detected characteristics.

### EXAMPLE 51

The method of Example 50 further comprising displaying the one or more detected characteristics.

### EXAMPLE 52

The method of any of one or more of Example 50 through Example 51 further comprising providing an alarm based on the one or more detected characteristics.

### EXAMPLE 53

The method of any one or more of Example 50 through Example 52, wherein the one or more characteristics includes one or more of a position, displacement, orientation, vibration, exposure, and temperature of the head fixation device.

### EXAMPLE 54

The method of any one or more of Example 50 through Example 53 further comprising modifying the head fixation device based on the one or more detected characteristics.

IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A device for stabilizing a patient comprising:
(a) a head fixation device comprising a first arm coupled with a first pin holder assembly and a second arm coupled with a second pin holder assembly; and
(b) one or more sensors positioned on the head fixation device, wherein the one or more sensors are configured to detect one or more characteristics of the head fixation device.

2. The device of claim 1, further comprising a data processing unit connected with the one or more sensors such that the data processing unit is configured to receive data from the one or more sensors, wherein the data processing unit is configured to analyze the characteristic detected by the one or more sensors to provide feedback based on the detected characteristic.

3. The device of claim 1, wherein the one or more characteristics of the head fixation device includes one or more of a position, displacement, orientation, load, and force of the head fixation device.

4. The device of claim 1, wherein the first pin holder assembly comprises a torque screw assembly including an actuator and a pin configured to contact a head, wherein the actuator is configured to adjust the pin relative to the head.

5. The device of claim 4, wherein at least one sensor is positioned on the actuator and is configured to detect one or more of a position, displacement, and angular orientation of the actuator.

6. The device of claim 4, wherein at least the one sensor is positioned adjacent the pin in a contacting configuration and is configured to detect one or more of a position, displacement, angular orientation, and force of the pin.

7. The device of claim 1, wherein the second pin assembly comprises a rocker arm and a pin coupled with the rocker arm, wherein at least one sensor is positioned on the pin and is configured to detect one or more of a position, displacement, angular orientation, and force of the pin.

8. The device of claim 2, wherein the data processing unit is configured to determine whether the fixation provided by the head fixation device is stable.

9. The device of claim 2, wherein the data processing unit is configured to compare the characteristic detected by the one or more sensors with a predetermined value to determine whether the fixation provided by the head fixation device is stable.

10. The device of claim 9, wherein the data processing unit is configured to provide an alarm to indicate when the fixation provided by the head fixation device is not stable.

11. The device of claim 2 further comprising a data interface connected with the data processing unit such that characteristic analyzed by the data processing unit is transmittable to the data interface.

12. The device of claim 11, wherein the data interface comprises a display for displaying the characteristic analyzed by the data processing unit.

13. The device of claim 11, wherein the data interface is configured to connect the data processing unit with remote devices.

14. The device of claim 11, wherein the data interface is configured to connect the data processing unit with a navigation system configured to display a diagram of the analyzed characteristic with a head of the patient.

15. The device of claim 11, wherein the data interface is configured to connect the data processing unit with an augmented reality system configured to display an image of the analyzed characteristic with a head of the patient.

16. The device of claim 1, further comprising a connection assembly connected with the sensor assembly, wherein the connection assembly comprises a data processing unit configured to receive data from the one or more sensors, wherein the connection assembly is configured to analyze the characteristic detected by the one or more sensors to provide feedback of the detected characteristic.

17. A method of operating a head stabilization system comprising a head fixation device having a first arm coupled with a first pin holder assembly and a second arm coupled with a second pin holder assembly, one or more sensors positioned on the head fixation device, and a data processing unit connected with the one or more sensors such that the data processing unit is configured to receive data from the one or more sensors, the method comprising the steps of:
(a) detecting one or more characteristics of the head fixation device; and
(b) analyzing the one or more detected characteristics.

18. The method of claim 17, further comprising providing an alarm based on the one or more detected characteristics.

19. The method of claim 17, wherein the one or more characteristics includes one or more of a position, displacement, orientation, load, and force of the head fixation device.

20. The method of claim 17 further comprising adjusting the head fixation device based on the one or more detected characteristics.
